(19) United States
(12) Patent Application Publication
(10) Pub. No.: US 2022/0238231 A1
(43) Pub. Date: Jul. 28, 2022

(54) SYSTEM AND METHOD FOR GENERATING SYNTHETIC LONGITUDINAL DATA

(71) Applicant: REPLICA ANALYTICS, Ottawa (CA)

(72) Inventors: Khaled EL EMAM, Ottawa (CA); Lucy Mosquera, Ottawa (CA); Cem Subakan, Ottawa (CA)

(21) Appl. No.: 17/648,902

(22) Filed: Jan. 25, 2022

Related U.S. Application Data
(60) Provisional application No. 63/141,282, filed on Jan. 25, 2021.

(51) Int. Cl. G16H 50/20; G06N 3/08; G16H 50/70

(52) U.S. Cl. CPC G16H 50/20; G16H 50/70; G06N 3/08

(57) ABSTRACT

Longitudinal data can be synthesized by first generating baseline characteristics and first event values for a plurality of synthetic individuals. The baseline characteristics and first event values are used to synthesize a plurality of subsequent events.

### RELATED APPLICATIONS

This application claims priority to US Provisional application 63/141,282, filed Jan. 25, 2021, entitled “SYSTEM AND METHOD FOR SYNTHESIZING LONGITUDINAL DATA” the entire contents of which is incorporated herein by reference in its entirety.

### TECHNICAL FIELD

The present disclosure relates to synthesizing a dataset, and in particular to synthesizing a dataset of longitudinal data.

### BACKGROUND

It is often difficult for analysts and researchers to get access to high quality individual-level health data for research purposes. For example, despite funder and journal expectations for authors to share their data, an analysis of the success rates of getting individual-level data for research projects from authors found that the percentage of the time these efforts were successful varied significantly and was generally low. Further, some researchers note that getting access to datasets from authors can take from 4 months to 4 years. Similarly, data access through independent date repositories can also take months to complete.

Concerns about patient privacy, coupled with increasingly strict privacy regulations, have contributed to the challenges noted above. There are a number of approaches that are available to address these concerns including consent, anonymization, and data synthesis.

While patient (re-)consent is one legal basis for making data available to researchers for secondary purposes, it is often impractical to get retroactive consent under many circumstances and there is risk of consent bias.

Anonymization is one approach to making clinical trial data available for secondary analysis. However, recently there have been repeated claims of successful re-identification attacks on anonymized data, eroding public and regulators' trust in this approach.

Data synthesis is another approach for creating non-identifiable health information that can be shared for secondary analysis by researchers. Researchers have noted that synthetic data does not have an elevated identity disclosure (privacy) risk, and recent empirical evaluations have demonstrated low risk. There are multiple methods that have been developed for the generation of cross-sectional synthetic health data. However, the synthesis of longitudinal data is more challenging.

An additional, alternative, new and/or improved method of synthesizing longitudinal datasets is desirable.

### BRIEF DESCRIPTION OF THE DRAWINGS

Further features and advantages of the present disclosure will become apparent from the following detailed description, taken in combination with the appended drawings, in which:

FIG. 1 depicts a representation of longitudinal health data;

FIG. 2 depicts a system for synthesizing longitudinal data;

FIG. 3 depicts details of an illustrative model for synthesizing longitudinal data;

FIG. 4 depicts a method for synthesizing longitudinal data;

FIG. 5 depicts a sequence length comparison between the real and synthetic datasets;

FIG. 6 depicts an event distribution comparison between the real and synthetic datasets;

FIG. 7 depicts the Hellinger distance for each event attribute;

FIG. 8 depicts heatmaps of first order Markov transition matrices between the real and synthetic datasets; and

FIG. 9 depicts adjusted hazard ratios for outcomes of interest in the synthetic data compared to the real data.

### DETAILED DESCRIPTION

In accordance with the present disclosure, there is provided a method for synthesizing longitudinal data comprising: generating baseline characteristics and first event values for a plurality of synthetic individuals using a trained model; for each synthetic individual in the generated baseline characteristics, generating a plurality of sequential event values by iteratively: using a trained model, predicting a next event comprising an event label and associated event attributes based on previous events for the respective synthetic individual; and masking from the predicted next event any predicted associated event attributes based on an attribute mask associated with the event label of the predicted next event; and outputting a synthetic data set comprising the synthesized baseline characteristics, first event values and synthesized sequential events of the plurality of synthetic individuals.

In a further embodiment of the method, the trained model for synthesizing the baseline characteristics and first event values uses a sequential tree-based method.

In a further embodiment of the method, the trained model used for predicting a next event comprises a long short term memory (LTSM) model.

In a further embodiment of the method, each event label is predicted from a predefined set of event labels.

In a further embodiment of the method, the trained model used for predicting a next event further comprises a first embedding layer for mapping event labels to a series of continuous features that are provided as input to the LTSM model.

In a further embodiment of the method, the trained model used for predicting a next event further comprises a second embedding layer for mapping event attributes to a series of continuous features that are provided as input to the LTSM model.

In a further embodiment of the method, each of the plurality of sequential events are associated with an event time of occurrence.

In a further embodiment of the method, the method further comprises training the model used to synthesize baseline characteristics and first event values from real longitudinal data.

In a further embodiment of the method, the method further comprises training the model used to synthesize the plurality of sequential event values using real longitudinal data.

In a further embodiment of the method, the longitudinal data comprises health data.

In accordance with the present disclosure there is further provided a non-transitory computer readable memory, which when executed configure a computing system to implement a method for synthesizing longitudinal data. The method comprising: generating baseline characteristics and first event values for a plurality of synthetic individuals using a trained model; for each synthetic individual in the generated baseline characteristics, generating a plurality of sequential event values by iteratively: using a trained model, predicting a next event comprising an event label and associated event attributes based on previous events for the respective synthetic individual; and masking from the predicted next event any predicted associated event attributes based on an attribute mask associated with the event label of the predicted next event; and outputting a synthetic data set comprising the synthesized baseline characteristics, first event values and synthesized sequential events of the plurality of synthetic individuals.

In a further embodiment of the non-transitory computer readable memory, the trained model for synthesizing the baseline characteristics and first event values uses a sequential tree-based method.

In a further embodiment of the non-transitory computer readable memory, the trained model used for predicting a next event comprises a long short term memory (LTSM) model.

In a further embodiment of the non-transitory computer readable memory, each event label is predicted from a predefined set of event labels.

In a further embodiment of the non-transitory computer readable memory, the trained model used for predicting a next event further comprises a first embedding layer for mapping event labels to a series of continuous features that are provided as input to the LTSM model.

In a further embodiment of the non-transitory computer readable memory, the trained model used for predicting a next event further comprises a second embedding layer for mapping event attributes to a series of continuous features that are provided as input to the LTSM model.

In a further embodiment of the non-transitory computer readable memory, each of the plurality of sequential events are associated with an event time of occurrence.

In a further embodiment of the non-transitory computer readable memory, the method provided by executing the instructions stored on the non-transitory computer readable memory further comprises training the model used to synthesize baseline characteristics and first event values from real longitudinal data.

In a further embodiment of the non-transitory computer readable memory, the method provided by executing the instructions stored on the non-transitory computer readable memory further comprises training the model used to synthesize the plurality of sequential event values using real longitudinal data.

In a further embodiment of the non-transitory computer readable memory, the longitudinal data comprises health data.

In accordance with the present disclosure, there is further provided a system for synthesizing longitudinal data comprising: a processor for executing instructions; and a memory storing instructions which when executed configure the system to implement a method for synthesizing longitudinal data, the method comprising: generating baseline characteristics and first event values for a plurality of synthetic individuals using a trained model; for each synthetic individual in the generated baseline characteristics, generating a plurality of sequential event values by iteratively: using a trained model, predicting a next event comprising an event label and associated event attributes based on previous events for the respective synthetic individual; and masking from the predicted next event any predicted associated event attributes based on an attribute mask associated with the event label of the predicted next event; and outputting a synthetic data set comprising the synthesized baseline characteristics, first event values and synthesized sequential events of the plurality of synthetic individuals.

As described further below, synthetic longitudinal patient data may be generated allowing data sets to be used without increased identification or privacy risks. Generating synthetic longitudinal data, such as longitudinal patient data, is challenging because patients can have long sequences of events that need to be incorporated into the generative models. Longitudinal data captures events and transactions over time, such as in electronic medical records, insurance claims datasets, and prescription records. Published methods thus far are not suitable for the synthesis of realistic longitudinal data because many of them only work with curated data where the messiness of real-world data has been taken out.

In generating synthetic longitudinal data it is desirable to have the characteristics of real longitudinal datasets that have received minimal curation to ensure that the synthesized datasets are realistic and that the generative models will work with real health data. Further it is desirable that the characteristics of the generative models themselves provide models that are scalable and generalizable. In order to address these desires, the model was developed to work with datasets that have real world characteristics. The assumed characteristics of these datasets are set forth further below.

The original dataset that is synthesized is a combination of (a) Longitudinal data (i.e. it has multiple events over time from that same patient) and (b) Cross-sectional data (i.e. it has measures that are fixed and are not repeated such as demographic information).The length of the longitudinal sequence varies across patients in the original datasets. Patients with acute conditions may have very few events, whereas complex patients with chronic conditions may have a very large number of events. The original datasets are heterogeneous with a combination of (a) Categorical or discrete features; (b) Continuous features; and (c) Categorical variables with high cardinality (e.g., diagnosis codes and procedure codes).Outliers and rare events should be retained in the original dataset since real data will have such events in them. The data may have many missing values, leading to sparse datasets (i.e., missing data are not removed from the original datasets that are synthesized).

In addition to the characteristics of the datasets, it is desirable that the generative model be able to take into account all of the previous information about the patients in the sequence. Further, it is desirable the generative model be developed based on existing data rather than requiring manual intervention by clinicians to seed it or correct it.

The model and process for generating synthetic longitudinal data described further herein meets the above noted desired characteristics of the generative model while using datasets in accordance with the desired characteristics.

As described further herein, a recurrent neural network based model (RNNs) may be used to generate synthetic longitudinal data from complex longitudinal health data or other types of longitudinal data. RNNs model input sequences using a memory representation which is aimed to capture temporal dependencies. Vanilla RNNs, however, suffer from the problem of vanishing gradients and thus, have difficulty capturing long-term dependencies that may be present in the longitudinal data. The current system and methods use long short-term memory units (LSTM) to model and synthesize observations over time. LSTM units, along with gated recurrent units (GRU) may be used to overcome the limitations of vanilla RNNs in generating synthetic longitudinal data.

In addition to generating the synthetic data, the generated synthetic data may also be evaluated in terms of data utility. The utility of the generated data can be evaluated using two approaches: general purpose utility metrics and a workload aware evaluation. The general purpose utility approach evaluates the extent to which the characteristics and structure of the generated synthetic data are similar to characteristics of the real data. The workload aware evaluation compares the model results and conclusions of a substantive analysis using the synthetic and real datasets. Both types of utility assessment are provided below.

A recurrent neural network model is described further below that was used for the generation of longitudinal health data from the province of Alberta. The utility of the generated synthetic data was evaluated. Utility may be considered as a measure of how similar the results and conclusions are from models built using the real data compared to the synthetic data.

The model used to generate the synthetic data was empirically tested on Alberta's administrative health records. Individuals were selected for this cohort if they received a prescription for an opioid during the 7-year study window. Data available for this cohort of patients included demographic information, laboratory tests, prescription history, physician visits, emergency department visits, hospitalizations, and death. The synthesized data utility was evaluated using generic metrics to compare the real data with the synthetic data, and a traditional time-to-event analyses on opioid use was performed on both datasets and the results compared. This type of analysis is the cornerstone of most health services research.

A cohort of patients previously derived and published to evaluate trends in opioid use in the province of Alberta, Canada was used in evaluating the synthesis of longitudinal data. The following administrative databases from Alberta Health from 2012 to 2018 were linked by the encrypted personal health number (PHN) for this cohort.
1) The Provincial Registry and Vital Statistics database for patient demographics and mortality. The age, sex, vital statistics, and date of last follow-up were used. An additional covariate was derived, the Elixhauser comorbidity score, based on physician, emergency department or hospitalization ICD-9/10 codes.
2) Dispensation records for pharmaceuticals from the Alberta Netcare Pharmaceutical

Information Network (PIN). The data was restricted to only dispensations of either one of two commonly dispensed opioids of interest in the data (morphine and oxycodone) and dispensations of antidepressant medications.
3) The Ambulatory Care Classification System which provides data on all services while under the care of the Emergency Department.
4) Discharge Abstract Database which provides similar data but pertaining to inpatient hospital admissions. Information on hospitalizations was restricted to the date of admission and the resource intensity weight, which is a measure used in the province to determine the amount of resources used during the stay. In addition, for hospitalizations, the primary diagnostic code according to ICD-10 coding within the hospital data was used to evaluate a cause specific event.
5) Provincial laboratory data which includes all outpatient laboratory tests in the province. 3 common labs conducted in the province (ALT, eGFR, HCT) were considered and the associated date of testing (first test ordered after start of follow up).

Although not used in the above noted cohort, additional information may be included in generating an evaluation cohort, including for example billing information associated with physician claims, such as may be available from, for example, Alberta Physician Claims Data.

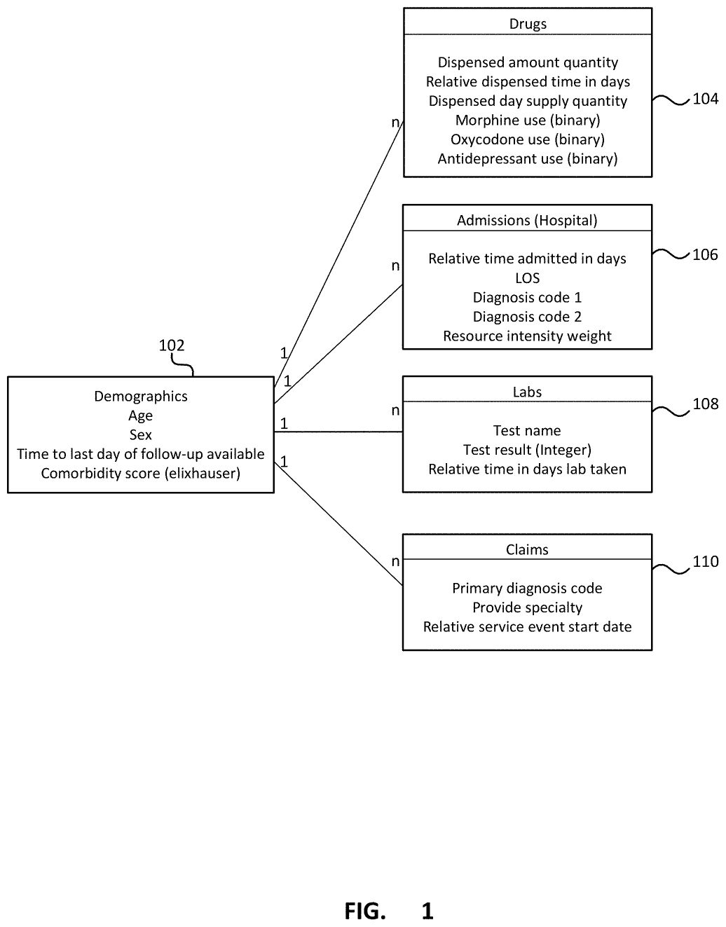

FIG. 1 depicts a representation of longitudinal health data. There is a demographic table or object 102 with basic characteristics of patients, and a set of transactional tables or objects including a drugs table or object 104, an admissions table or object 106, a labs table or object 108, and a claims table or object 110. The demographics information contains a single observation per individual, where each individual is identified using a personal health number (PHN). This PHN links the demographics table to all other tables in the dataset, where all other tables may have multiple observations per individual. Each of the transactional tables or objects 104-110 have a one-to-many relationship between the demographic table and the transactional tables. Therefore, each patient may have multiple events occurring over time. Using the PHN, observations for a single individual from multiple transactional tables may be grouped together. Each observation in the transactional tables includes the date of the event relative to the start of the study period. This means that a group of observations from the same individual may be sorted according to the relative date, yielding a chronological set of an individual's interactions with the health system. It will be appreciated that additional data not depicted in FIG. 1 may be included if records for individual patients can be linked together, such as by using the PHN. For example, data on physician visits may be included.

Each event, whether it is a visit or a lab test, or some other event has a different set of attributes. Therefore, the event characteristics are a function of the event type. For example, a hospitalization event will record the relative date of the hospitalization, the length of stay, diagnostic code, and a metric for resource utilization. Additionally, all event types include an attribute to describe the timing of the event. The current process models time using sojourn time, or time in days since the last event for that individual.

The basic patient characteristics and event characteristics are heterogeneous in type. This means that some will be categorical variables, some will be continuous, some binary, and some discrete ordered variables. For example, age is a continuous patient characteristic while diagnostic code associated with an emergency department visit is a categorical event characteristic.

Table 1 provides the exact dimensionality of the original datasets. A random subset of 100,000 patients from a population of 300,000 subjects who received a dispensation for morphine or oxycodone between Jan. 1, 2012 and Dec. 31, 2018, 18 years of age and over were included in the analyses presented herein. For these patients, the events were truncated at the 95th percentile, which means that the maximum number of events that an individual can have was 1000.

TABLE 1
Dimensionality of the original data tables for the approximately
100,000 individuals used for training.

| Table Name | Number of Rows | Number of Columns |
|---|---|---|
| Age_sex_comorbidity | 100,000 | 4 |
| Drug_data | 9,975,950 | 7 |
| ED_visits | 1,748,083 | 5 |
| Hosp_admit | 84,669 | 5 |
| Labs | 2,199,574 | 3 |
| MD_claims | 8,538,816 | 4 |
| Reg_file | 100,000 | 2 |
| Vital_stats | 4,200 | 6 |

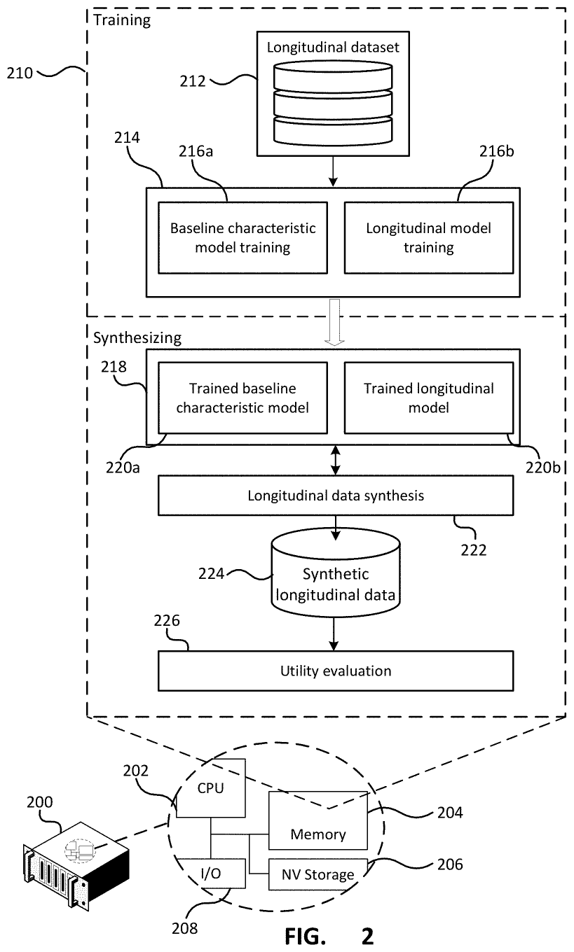

FIG. 2 depicts a system for synthesizing longitudinal data, such as the dataset described above. The system 200 is depicted as a single server, however the functionality may be provided by one or more servers. The system 200 comprises a processor (CPU) 202 for executing instructions and a memory 204 for storing data and instructions that can be executed by the processor to configure the system to provide various functionality. The system 200 may further comprise non-volatile storage 206 and an input/output (I/O) interface 208 for connecting one or more devices or components to the system such as a graphics processing unit (GPU). GPU may be well suited for processing on a GPU instead of the CPU. It will be appreciated that the processes described herein may be performed on the GPU, CPU or both. The data and instructions stored in the memory may be executed by the processor 202 to configure the system to provide training and synthesizing functionality 210.

The functionality 210 includes training functionality that uses a real longitudinal dataset 212 to train models used in synthesizing corresponding data. The functionality 210 includes synthetic data generation model training functionality 214 that may comprise baseline characteristic model training functionality 216a, that trains a model used in synthesizing baseline characteristics for individuals. The synthetic data generation model training functionality 214 may further comprise longitudinal model training functionality 216b trains a model that can be used to synthesize longitudinal data.

The synthetic data generation model training functionality 214 trains a synthetic data generation model 218 that may comprise, for example, a baseline characteristic model 220a and a longitudinal model 220b respectively. Longitudinal data synthesis functionality 222 may use the synthetic data generation model 218, including both the trained baseline characteristic model 218 and the longitudinal model 220 to generate synthetic longitudinal data 224. The synthetic longitudinal data may be generated by first using the trained baseline characteristic model 218 to synthesize starting information and then using the trained longitudinal model to iteratively synthesize longitudinal event data from the generated starting information. Utility evaluation functionality 226 may be used to evaluate the generated longitudinal data 224. The utility evaluation may be used to adjust the data synthesis if the evaluated utility does not meet a desired level. Further, although not depicted, the privacy or re-identification risk of the generated synthetic data may also be evaluated. The privacy evaluation may also be used, possibly in conjunction with the utility evaluation to adjust the data synthesis in order to balance a desired privacy level with the utility of the synthetic data.

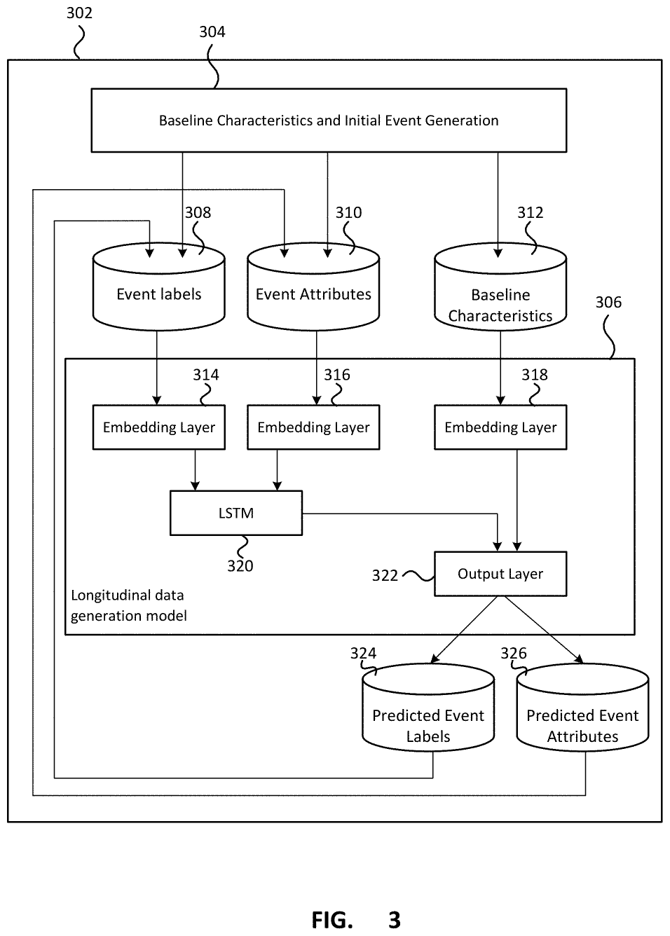

FIG. 3 depicts details of an illustrative model for synthesizing longitudinal data. FIG. 3 provides a diagram of an overall RNN architecture. The machine learning model 302, which may be used as the trained synthetic longitudinal data generation model 218 described above with reference to FIG. 2, is used to describe and generate new synthetic datasets. The machine learning model 302 comprises a baseline characteristics and initial event generation model 304 which generates the initial input for a longitudinal data generation model 306. The baseline characteristics and initial event generation may be generated in various ways, including for example randomly sampling starting values; however, using a sequential tree-based synthesis approach may produce synthetic values for the baseline characteristics and starting values for the event labels and attributes that better reproduce the characteristics of the real population.

The longitudinal data generation model 306 may be a form of LSTM where the final predicted outputs are conditional on the baseline characteristics. The input data corresponds to n individuals at t−1 time points (e.g., the set t=1, 2, 3, . . . t−1) for event labels 308 (yielding an array of dimensions [n, t−1]) and event attributes 310 (yielding an array of dimensions [n, t−1,A] where A is the number of attributes) as well as the B baseline characteristics 312 for each individual. The event labels and event attributes are iteratively predicted based on previous event labels and attributes. The output comprises predictions corresponding ton individuals at t−1 time points (e.g., the set t=2, 3, 4, . . . t) for the event labels 324 and event attributes 326. These predictions may be used during training to calculate the model loss, or during data generation as the subsequent synthetic events.

While the event labels 308 and event attributes 310 and the predicted event labels 324 and predicted event attributes 326 are the same dimension, event labels 308 and event attributes 310 correspond to times t=1,2,3, . . . t−1 within the real data while the predicted event labels 324 and predicted event attributes 326 correspond to times t=2,3,4, . . . t. As depicted in FIG. 3, the machine learning model used to describe and generate the synthetic longitudinal data is a form of LSTM where the final predicted outputs are conditional on the baseline characteristics.

The input data corresponds to n individuals at t−1 time points (e.g., the set t=1,2, 3, . . . t−1) for event labels (yielding an array of dimensions [n, t−1]) and event attributes (yielding an array of dimensions [n, t−1,A] where A is the number of attributes) as well as the B baseline characteristics for each individual (yielding an array of [n, B] where B is the number of baseline characteristics). The output comprises predictions corresponding to n individuals at t−1 time points (e.g., the set t=2, 3, 4, . . . t) for the event labels and event attributes. These predictions are used during training to calculate the model loss, or during data generation as the subsequent synthetic events. The event data may be provided in various formats, including for example as two tensors, one of dimension [n, t] corresponding to the event labels for n individuals at t time points, and the other of dimension [n, t, A] where A corresponds to the number of event attributes.

The longitudinal data generation model is depicted as comprising three embedding layers 314, 316, 318 for the event labels, event attributes and baseline characteristics respectively; an LSTM 320 connected to the event label and event attributes embedding layers; and an output layer 322. The embedding layers 314, 316, 318 may be used to map single integer encoded categorical features to a series of continuous features. The benefit of this embedding is that the transformation to map the discrete features to the set of continuous features is altered and improved throughout training. This allows for a continuous space representation of the categorical features that picks up similarity between related categories. Embedding occurs independently for each of the baseline characteristics (age, sex, comorbidity index), the event labels, and the event attributes.

The LSTM 320 estimates a representation of the hidden state given the prior event labels and attributes. The embedded event attributes and the embedded event labels may be concatenated prior to being input in the LSTM. If the LSTM receives observations corresponding to times tϵ{1, 2, 3, . . . t−1}, then the output of the hidden state will correspond to times tϵ{2, 3, 4, . . . t}. In addition to the predictions, the LSTM outputs the complete hidden state which describes the current state of all elements of the model. The complete hidden state may be used during data synthesis as a way of accounting for historical events.

The output layer 322 may comprise a set of linear transformations that take as input the concatenation of the output of the LSTM and the embedded baseline characteristics. The output layer 322 make the predictions for the next time points generated by the LSTM conditioned on the baseline characteristics.

The longitudinal data generation model may be trained in various ways. One example of training a model is described further below.

During training, loss may be calculated using cross entropy. For each individual at each time point, cross entropy loss can be calculated between the predicted event labels and the true event labels, then these values are averaged:

$${loss}_{labels} = \frac{1}{N t} {\sum }_{n = 1}^{N} {\sum }_{t = 1}^{t} - x l a b e {l}_{n , t} [ {true}_{n , t} ] + log ( {\sum }_{j = 0}^{C} exp ( x l a b e {l}_{n , t} [ j ] ) )$$

Where xlabel_{n,t} is the vector of predicted probabilities for the event label for individual n at time t where xlabel_{n,t}[j] is the predicted probability that individual n at time t has event j. true_{n,t} is the true event label for individual n at time t. Then, cross entropy loss is calculated for the attributes associated with the true event label. For example, if the next time point is truly a lab test, then the model loss for the event attributes is the sum of the cross entropy between the real lab test name and the predicted lab test name and the cross entropy between the real lab test result and the predicted lab test result. This masked form of loss for the event attributes is desirable as it allows the model to focus on learning the relevant features at each time point, rather than constantly predicting missing values.

If the indicator function is defined as 1(Aᵢ|true_{n,t}) returning 1 if a given attribute Aᵢ, is relevant for a given true event label true_{n,t, and} 0 otherwise; then cross entropy loss for the attributes may be calculated as:

$${loss}_{attributes} = mean \{ {\sum }_{n = 1}^{N} {\sum }_{t = 1}^{t} {\sum }_{i = 1}^{A} 1 ( {A}_{i} \vert {true}_{n , t} ) [ - {x}_{n , t , i} [ {true A}_{i , n , t} ] + log ( {\sum }_{j = 0}^{c} exp ( {x}_{n , t , i} [ j ] ) ) ] \}$$

Where trueA_{i,n,t} is the true value for individual n′s attribute i at time t and x_{n,t,i} is the vector of the predicted probabilities for individual n′s attribute i at time t among the C possible classes for attribute i.

Thus, the objective function for training is to minimize the total loss over the model parameters θ, where the tradeoff parameter controls the relative importance of label loss and attribute loss:

$${min}_{θ} \{ {loss}_{labels} + {λ loss}_{attributes} \}$$

Additionally or alternative, if the longitudinal data is continuous, training loss can be calculated using negative log probability. For this, each continuous feature is modelled using a probability distribution (e.g., normal distribution for unbounded, standardized variables, or beta distribution for bounded variables). The output layers then predict the model parameters for a given individual i at a time t. For example, for a variable v that is modelled using a normal distribution, the output layer will predict a mean μᵢₜ^{v} and a standard deviation σᵢₜ^{v}. During training, loss is then calculated using the log probability of observing attribute value Aᵢₜ^{v} given the predicted probability distribution N(μᵢₜ^{v}, σᵢₜ^{v}). This can be generalized to any two parameter (denoted: θᵢₜ^{v1} and θᵢₜ^{v2}, respectively), probability distribution D as -log (P(Aᵢₜ^{v}|D(θᵢₜ^{v1, θ}ᵢₜ^{v2}))This is then averaged and masked in a similar fashion as described above, yield the attribute loss function:

$${loss}_{attributes} = mean \{ {\sum }_{n = 1}^{N} {\sum }_{t = 1}^{t} {\sum }_{i = 1}^{A} 1 ( {A}_{i} \vert {true}_{n , t} ) [ - log ( P ( {A}_{it}^{v} \vert D ( {θ}_{it}^{v 1} , {θ}_{it}^{v 2} ) ) ] \}$$

This loss function allows the synthesis model to be trained on longitudinal data with continuous features and may be combined with the loss function for categorical longitudinal features.

During training, data may be provided for the model in tensors of 120 time points. Individuals have their data grouped into chunks of up to 120 sequential events with 0s introduced to pad chunks shorter than 100 observations. This is desirable as it produces data that is uniform and much less sparse than if the data were to be padded up to the true maximum number of observations per individual of 1000.

Hyperparameter optimization was performed using a training set of 100,000 individuals and a validation set of 20,000 individuals. Hyperparameters explored include batch size, number of training epochs, optimization algorithm, learning rate, number of layers within the LSTM, hidden size of the LSTM, embedding size for the event labels, event attributes, and baseline characteristics, and weighting for the different event types and event attributes during calculation of the training loss. Training was performed on an Nvidia® P4000 graphics card and was coordinated using Ray Tune.

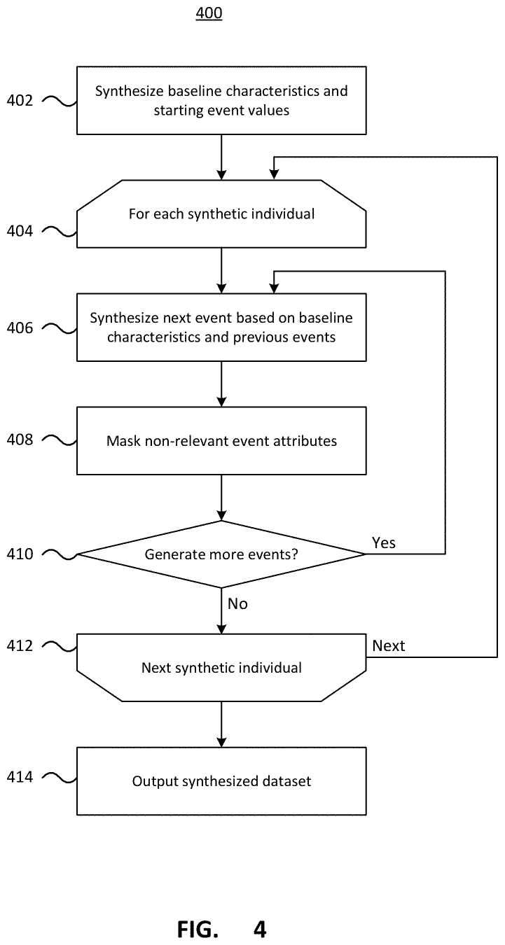

FIG. 4 depicts a method for synthesizing longitudinal data. After training the model as described above, synthetic data generation method 400 includes two phases: generation of baseline characteristics and starting values followed by the generation of event data. Baseline characteristics and values for the first event observed are generated (402) using for example a sequential tree-based synthesis model. Using a scheme similar to sequential imputation, trees are used quite extensively for the synthesis of health and social sciences data. With these types of models, a variable is synthesized by using the values earlier in the sequence as predictors.

For each of the synthetic individuals (404, 412), these synthesized values for the baseline characteristics and first event are then fed into the trained model to generate the remaining events for each synthetic individual. The goal behind using sequential tree-based synthetic values as the baseline characteristics and starting values for the LSTM model is that they will better reproduce the characteristics of the real population than randomly sampled starting values.

To generate the longitudinal event data, the output of the sequential tree-based synthesis is iteratively fed into the LSTM model. At each iteration, the model uses the synthetic data from the previous time point, as well as the hidden state of the model if available, to predict the next time point (406). These predictions comprise predicted event labels and event attributes. Based on the predicted event label, all non-relevant event attributes are masked (408), for example by setting the value to missing. A respective attribute mask may be associated with each possible event label. The attribute mask specifies which event attributes are ‘important’ or should be retained. The other attributes not masked may be considered as junk and either ignored or set to missing. For example, if the next time point predicts an event of lab tests, the lab test name, lab test result, and sojourn time event attributes will be retained while all others are set to missing. This masking during data generation helps to ensure that the data the model sees during data generations matches the format of the data seen during training. Data synthesis proceeds in this iterative fashion (Yes at 410) until the model has generated event data up to the maximum sequence length or other determination indicative that no more events need to be synthesized (No at 410). The next synthetic individual (412) may be processed. Although depicted as processing each individually one after the other, it is possible to processes synthetic individuals in parallel. Once the dataset is generated, it is output (414) and may be further processed. For example, splitting the synthetic sequence data into the original source data tables.

To improve the results of synthetic data generation for categorical longitudinal features, alternative sampling schemes may be deployed. During data generation for categorical longitudinal features, the synthesis model predicts a probability distribution for the classes within variable v. This multinomial distribution can be defined P(Aᵢₜ^{v}=Cⱼ)=pⱼ for all j classes. The default behavior is to sample from this distribution to generate the synthesized value for Aᵢₜ^{v}. However, this may lead to poor performance, especially when variables have high cardinality

Performance may be improved by implementing top-p sampling. Top-p sampling sorts the predicted probability distribution P(Aᵢₜ^{v}=Cⱼ)=pⱼ from largest to smallest pⱼ values, and then truncates the predicted probability distribution once the cumulative probability has reached a threshold. The remaining classes in the probability distribution are then reweighted, and sampled from.

In testing and evaluating the synthesis technique the original dataset was preprocessed. The main steps of data pre-processing may be broadly grouped as modifying the data structure and variable encoding. The goal of modifying the data structure is combining the different original data tables into a format that is suitable for the RNN. In contrast, variable encoding aims to format each variable in the dataset in a manner that is suitable for the RNN.

The original structure of the data provided had multiple forms linked by a single subject identifier where each form had a single type of health information. The goal of modifying the data structure is to transform these tables into a consistent representation for the machine learning model. Data was grouped based on whether they are longitudinal events that occur over time, compared to baseline characteristics.

In this dataset the baseline characteristics include the age, sex, and baseline comorbidity index for the individual. Additionally, the relative date of the individual's first observation is included as a baseline characteristic. These measures are then combined in a single dataset BC=[n,B] that has the following structure:

TABLE 2
Structure of baseline characteristic (BC) data.

| Encrypted | | | | Date of |
|---|---|---|---|---|
| PHN | Age | Sex | Comorbidity | First Obs |
| 10000001 | 38 | F | 0 | 100 |
| 10000002 | 22 | M | 0 | 325 |
| 10000003 | 70 | F | 1 | 52 |
| 10000004 | 55 | F | 0 | 89 |
| 10000005 | 63 | M | 3 | 600 |

The grouping depicted in Table 2 produces a table of size BC=[n, B], where n corresponds to the number of individuals in the dataset and B corresponds to the number of baseline characteristics present in the data. In this case B=4.

Longitudinal events include prescriptions, physician visits, hospitalizations, emergency department visits, and. These observations were joined from different data tables by assigning event type labels and associated attributes for each event type. For example, all observations from the hospitalization form are considered the event ‘hospitalization’ and have measures for the attributes such as, for example: length of stay and resource intensity weight. Given that not every attribute is measured for every event type, this yields a sparse data frame with many missing values for event attributes. Table 3 illustrates the structure of the joined data frame. This data frame captures all events that occur throughout the study period for each patient.

TABLE 3
Structure of joined longitudinal dataset for a single patient.

| | | | | | ICD10 | | | | ICD9 | Lab | Lab |
|---|---|---|---|---|---|---|---|---|---|---|---|
| Encrypted | | Sojourn | Amt | Duration | Diagnostic | | | Specialist | Diagnostic | Test | Test |
| PHN | Label | Time | Dispensed | of RX | Code | RIW | LOS | Type | Code | Name | Results |
| 1000001 | GP Visit | 0 | NA | NA | NA | NA | NA | NA | 311 | NA | NA |
| 1000001 | Other RX | 0 | 10 | 7 | NA | NA | NA | NA | NA | NA | NA |
| 1000001 | Antidep RX | 0 | 100 | 60 | NA | NA | NA | NA | NA | NA | NA |
| 1000001 | MD Visit | 62 | NA | NA | NA | NA | NA | ORTH | 724.5 | NA | NA |
| 1000001 | Morphine RX | 0 | 30 | 60 | NA | NA | NA | NA | NA | NA | NA |
| 1000001 | Lab Test | 2 | NA | NA | NA | NA | NA | NA | NA | GFR | 85 |
| 1000001 | GPVisit | 180 | NA | NA | NA | NA | NA | NA | 724.5 | NA | NA |
| 1000001 | Morphine RX | 0 | 60 | 60 | NA | NA | NA | NA | NA | NA | NA |
| 1000001 | ED Visit | 5 | NA | NA | N20.0 | 0.001 | NA | NA | NA | NA | NA |
| 1000001 | Hospitalization | 10 | NA | NA | 175.81 | 0.05 | 7 | NA | NA | NA | NA |
| 1000001 | Oxycodone RX | 0 | 120 | 7 | NA | NA | NA | NA | NA | NA | NA |
| 1000001 | Death | 7 | NA | NA | 175.81 | NA | NA | NA | NA | NA | NA |
| 1000001 | Last Obs | 0 | NA | NA | NA | NA | NA | NA | NA | NA | NA |

All original data tables correspond to a single event type (e.g., the hospitalization form yield ‘hospitalization’ events), except for the drug_data and MD claims forms. These two forms have 47 million and 29 million observations respectively, which constitutes 83% of the total number of event observations. To prevent strong imbalance between different event types, the drug_data form was split into 4 event types: morphine dispensations, oxycodone dispensations, antidepressant dispensations, and other prescription dispensations while the MD claims form was split into 2 event types: general practitioner visits and specialist visits. This split leverages the existing features in the data.

After joining observations from the different transactional tables, relative dates for each event were recoded as time between events or sojourn time. This transformation was conducted as longitudinal health data is often utilized for time to events type analyses, and therefore the modelling described herein prioritized the time between events rather than the relative dates of observations.

One important characteristic of this dataset is the wide range in the number of observations associated with each individual. Summarized as percentiles in Table 4, it is seen that most patients have dozens or hundreds of events recorded, while very few (<5% of patients) have between 1,000 and 36,774 events recorded. This great range in number of events is something that is desired to be preserved in the generated synthetic longitudinal dataset, that also may be associated with the features of the data itself (i.e. individuals with more observations may be sicker so they are more likely to have ongoing prescriptions, chronic conditions, etc.). For simplicity, patients with >1000 observations were omitted from the dataset, which is a cut at the 95^{th} percentile of event counts as shown in Table 4.

TABLE 4
Percentiles for the number of events per patient.

| Percentile | 0% | 5% | 10% | 15% | 20% | 25% | 30% | 35% | 40% | 45% | 50% |
|---|---|---|---|---|---|---|---|---|---|---|---|
| # Obs | 2 | 25 | 40 | 54 | 69 | 84 | 99 | 116 | 134 | 153 | 175 |

| Percentile | 55% | 60% | 65% | 70% | 75% | 80% | 85% | 90% | 95% | 100% |
|---|---|---|---|---|---|---|---|---|---|---|
| # Obs | 199 | 227 | 260 | 299 | 349 | 414 | 507 | 660 | 997 | 36774 |

For the formatted datasets described in Table 2 and Table 3 to be suitable for the RNN, feature encoding must occur. Feature encoding helps ensure that all features the model is attempting to learn are on similar scales. When minimizing error in prediction, features with larger ranges and thus larger prediction errors will be prioritized during training. This is not a desirable trait as it is desirable for each feature to be prioritized equally unless specified otherwise. For the LSTM models being applied, in order to make the training process easier, all features are discretized.

The kind of feature encoding performed depends on the format of the original variable. In this dataset the following transformation were performed:
Categorical variables with 100 levels: (e.g., lab test name, specialist type, event labels) were mapped 1 to 1 from the text categories to the integers 1, 2, 3, etc.
Continuous variables: (e.g., sojourn time, dispensed amount, prescription duration, length of stay, resource intensity weight, lab test result) were binned and then mapped to the integers 1, 2, 3, etc.
Categorical variables with >100 levels: (e.g., ICD9 and ICD10 diagnostic codes) were formatted based on prevalence in the data. Levels with many observations were kept in their original format, while levels that were less common were generalized to the chapter level.
Baseline characteristics were left in their original format, except for date of first observation. Date of first observation was scaled based on the study period (i.e., if the first observation for an individual was recorded on day 200, this was transformed using the 7 year, or 2557 day, study period to be

$$\frac{200}{2557} = 0.078 ) .$$

The synthetic data can be evaluated to determine its utility. Generic utility assessments aim to assess the similarity between a real and synthetic dataset without any specific use case or analysis in mind. Two types of methods were used depending on whether the utility of the cross-sectional or the longitudinal portion of the data were being evaluated.

### Event Distribution Comparisons

The simplest generic utility assessments are to compare the number and distribution of events generated for each synthetic individual to the number and distribution of events in the real data. To compare the number of events per individual, the distributions are plotted as histograms and the means are compared. To compare the distribution of events in the real and synthetic data, the observed probability distribution for event types is calculated for each dataset. This corresponds to what proportion of events belongs to each event type. These probability distributions are then plotted and compared as bar charts.

Additionally, these distributions are compared by calculating the Hellinger distance between the two distributions. Hellinger distance is an interpretable metric for assessing the similarity of probability distributions that is bounded between 0 and 1 where 0 corresponds to no difference.

### Comparing the Distribution of Event Attributes

Another simple metric for assessing the similarity between the real and synthetic datasets is to compare the distributions of each event attribute. For this assessment, the Hellinger distance (as defined above) is applied to the discrete probability distributions for each event attribute. For this assessment, careful consideration is taken to tabulate the probability distributions for each event attribute, only using observations with an event label that is relevant for that attribute. This ensures that comparisons are made between the distributions of each attribute without the padded/missing values. To summarize the Hellinger distance values calculated for each event attribute, they are plotted in a bar chart.

### Comparison of Transition Matrices

The next method applied for the utility evaluation of synthetic data was to compute the similarity between the real data and the synthetic data transition matrices. A transition matrix reflects the probability of transitioning from one event to another. These transition probabilities can be estimated empirically by looking at the proportion of times that a particular event follows another one.

For example, consider sequence data with four events: A, B, C, and D where C is a terminal event, meaning that C if occurs, a sequence terminates. If 40% of the time an event B follows an event A, then it is possible to say that the transition from A to B has a probability of 0.4. The transition matrix is the complete set of these transition probabilities. Creating such a transition matrix assumes that the next event observed is dependent on only one previous event. This can be quite limiting and does not account for longer term relationships in the data. However, transition matrices can be extended to the k^{th} order where k corresponds to the number of previous events considered when calculating the transition probabilities.

An example of a 2^{nd} order transition matrix is shown in Table 5. There are two previous events along with the transition probabilities. The rows indicate the previous states, and the columns indicate the next state. Note that each row needs to add up to 1 because the sum of the total transitions from a pair of consecutive states must be 1. Also, there are no previous states with a C event in them because in the example that is a terminal event.

TABLE 5
An example of a transition matrix with an order of
2, which means that the two previous events are considered.
It is assumed that C is a terminal event.

| | A | B | C | D |
|---|---|---|---|---|
| AB | 0.31 | 0.29 | 0.39 | 0.00 |
| BA | 0.42 | 0.21 | 0.22 | 0.16 |
| AD | 0.64 | 0.11 | 0.08 | 0.18 |
| DA | 0.38 | 0.05 | 0.23 | 0.34 |
| BD | 0.41 | 0.31 | 0.26 | 0.02 |
| DB | 0.01 | 0.16 | 0.57 | 0.26 |
| AA | 0.20 | 0.40 | 0.30 | 0.10 |
| BB | 0.36 | 0.34 | 0.25 | 0.04 |
| DD | 0.34 | 0.48 | 0.17 | 0.01 |

The transition matrices for the real and synthetic datasets can be compared by calculating the Hellinger distance between each row in the real transition matrix and the corresponding row in the synthetic transition matrix. The lower the Hellinger distance values, the closer the transition structure between the two datasets. The utility for both the 1^{st} and 2^{nd} order transition matrices are provided.

### Comparison of Graph Structure

The last method that was applied for generic utility evaluation was to convert each longitudinal record into a directed graph then comparing the sample of real and synthetic graphs to test if they come from similar underlying distributions. This utility assessment aims to see if the synthetic patient records are like the real records in terms of the numbers and progressions of events observed. For each patient record, the longitudinal data is transformed into a graph where each event type will be treated as a node (e.g., hospitalization, lab test, prescription, and so on). If a patient went to the hospital first and then took a lab test, there will be a directed edge from the hospital node to the lab test node. In addition, if this transition happens N times, then it is possible to label this directed edge as N to capture the number of times this transition occurs. Therefore, the graph for each longitudinal record is a directed graph with edges labeled by how many times event A occurs after event B, for all combinations of events.

A traditional way to measure the similarity of two datasets is called Maximum Mean Discrepancy (MMD). The main idea of the MMD is that if two datasets have the same distribution the squared difference of the statistics between the two sets of samples should be small [58][59].

Given a kernel K: X×Y → [symbol] , and samples {xᵢ}ᵢ₌₁^{N} and {yⱼ}ⱼ₌₁^{M}, an unbiased estimate of MMD² is:

$${u}_{2}^{} = \frac{1}{n ( n - 1 )} {\sum }_{i = 1}^{n} {\sum }_{j \neq i}^{n} K ( {x}_{i} , {x}_{j} ) - \frac{2}{mn} {\sum }_{i = 1}^{n} {\sum }_{j = 1}^{m} K ( {x}_{i} , {y}_{j} ) + \frac{1}{m ( m - 1 )} {\sum }_{i = 1}^{m} {\sum }_{j \neq i}^{m} K ( {y}_{i} , {y}_{j} )$$

However, since the data is represented as graph, a popular approach to learning with graph-structured data is to make use of graph kernels—functions which measure the similarity between graphs—plugged into a kernel machine, such as a support vector machine.

It is possible to calculate the MMD using the edge histogram kernel, which is a basic linear kernel on edge label histograms. The kernel assumes edge-labeled graphs, which is exactly the case for the dataset. Let

[symbol]

be a collection of graphs and assume that each of their edges comes from an abstract edge space ϵ. Given a set of node labels [symbol] [symbol] ϵ→ [symbol]
is a function that assigns labels to the edges of the graphs. Assume that there are d labels in total, that is d=| [symbol] |. Then, the edge label histogram of a graph G=(V, E) is a vector f=(f1, f2, . . . , fd). such that fᵢ=|{(v,u)ϵE: [symbol] (v,u)=i}| for each iϵ [symbol]
Let f, f′ be the edge label histograms of two graphs G, G′, respectively. The edge histogram kernel is then defined as the linear kernel between f and f′, that is: k(G,G′)=f, f′>[60].

### Analysis Specific Utility Assessments

Generic utility assessments are agnostic to the future analyses of the synthetic data and compare the real and synthetic datasets in terms of distributional and structural similarity. In contrast, workload aware or analysis-specific utility assessments compare the real and synthetic datasets by applying the same analysis to both and comparing the results. For this dataset an analysis-specific utility assessment was conducted by applying a common analytical approach used in time to event analyses in administrative health data to both the real and synthetic datasets and comparing the results.

The primary outcome was a composite endpoint of all-cause emergency department visit, hospitalization, or death during the follow-up. The secondary outcomes included each component of the composite endpoint separately, as well as to evaluate cause specific admissions to hospital for pneumonia (J18) as a prototypical example of a cause specific endpoint.

First, all variables in both the synthetic and real data were compared using standard descriptive statistics (e.g., means, medians). Second, standardized mean differences (SMD) were used to statistically compare the variables of interest between the synthetic and real data. SMD was selected as given the large sample size, small, clinically unimportant differences, are likely to be statistically different when using t-tests or chi squared test. A SMD greater than 0.1 is deemed as a potentially clinically important difference, a threshold often recommended for declaring imbalance in pharmacoepidemiologic research.

Using Cox proportional hazards regression models, unadjusted and adjusted hazard ratios (HRs) and 95% CIs were calculated to assess the risk associated with either morphine or oxycodone and the outcomes of interest in both the synthetic and real data separately. Start of follow-up began on the date of the first dispensation for either morphine or oxycodone. All subjects were prospectively followed until outcome of interest or censoring defined as the date of termination of Alberta Health coverage or 31 March 2018, providing a maximum follow-up of 7 years. Finally, the estimates derived from the real and synthetic datasets were directly statistically compared. Morphine served as the reference group for all estimates. Potential confounding variables included in all multivariate models included age, sex, Elixhauser comorbidity score, use of antidepressant medications, and the 3 laboratory variables (ALT, eGFR, HCT). To compare the confidence intervals estimated for HRs from real vs synthetic dataset, confidence interval overlap was used. All analyses were performed using STATA/MP 15.1 (StataCorp., College Station, Tex.).

In testing the data synthesis, hyperparameter training was conducted for a variety of aspects of model implementation. By selecting the values within a search range that minimized validation loss, the optimal models were selected for the two variants of the dataset. A set of values for the hyperparameters as selected by hyperparameter optimization for generating each of the synthetic datasets is provided in Table 6. The hyperparameter optimization was performed on an Nvidia® p4000 GPU.

TABLE 6
Optimal model parameters as selected
via hyperparameter optimization

| | Optimal Value |
|---|---|
| Batch Size | 256 |
| Training Epochs | 50 |
| Learning Rate | 8.98 × 10⁻⁶ |
| Optimization | ADAM |
| Algorithm | |
| LSTM Layers | 1 |
| LSTM Hidden Size | 648 |
| Embedding Size | [sex: 3, elixhauser: 9, age: 13] |
| for Baseline | |
| Characteristics | |
| Embedding Size for | 29 |
| Event Labels | |
| Embedding Size for | [sojourn time: 8, dispensed amount: 12, dispensed |
| Event Attributes | days: 12, ED diagnostic code: 18, ED RIW: 12, |
| | hospitalization length of stay: 12, hospitalization |
| | diagnostic code: 8, hospitalization RIW: 12, cause |
| | of death: 12, lab test name: 9, lab test result: 12] |

The generic utility results for the complete data are summarized in Table 7, and are reviewed in more detail below.

TABLE 7
Summary of the generic utility assessments results.

| Metric | Result | |
|---|---|---|
| Percent difference in sequence lengths | 0.4% | |
| Hellinger distance of event distribution | 0.027 | |
| Hellinger distance of event attributes | | |
| Mean (SD) | 0.0417 | |
| Median (IQR) | 0.0303 | (0.0333) |
| Hellinger distance of Markov Transition | | |
| Matrices of Order 1: | | |
| Mean (SD) | 0.0896 | (0.159) |
| Median (IQR) | 0.0209 | (0.0303) |
| Hellinger distance of Markov Transition | | |
| Matrices of Order 2: | | |
| Mean (SD) | 0.2195 | (0.2724) |
| Median (IQR) | 0.0597 | (0.4401) |

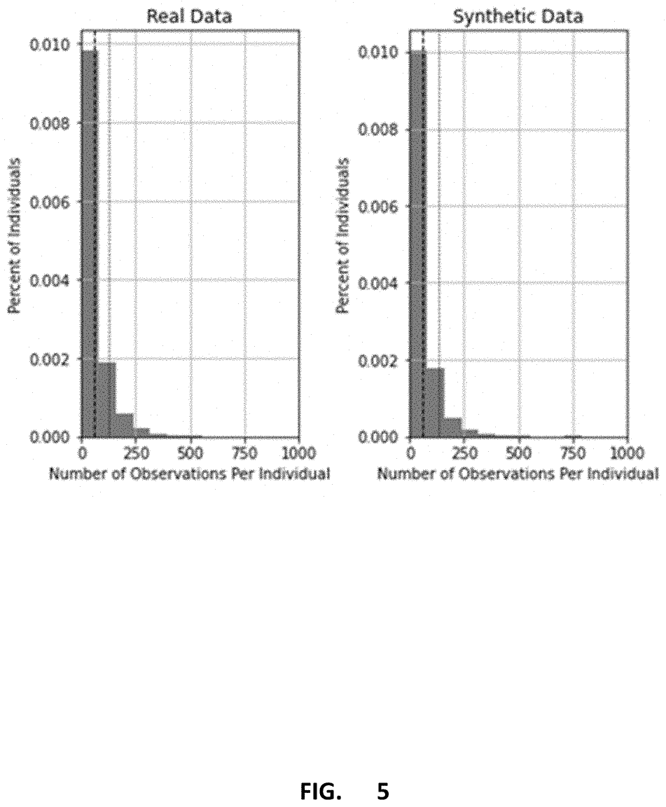

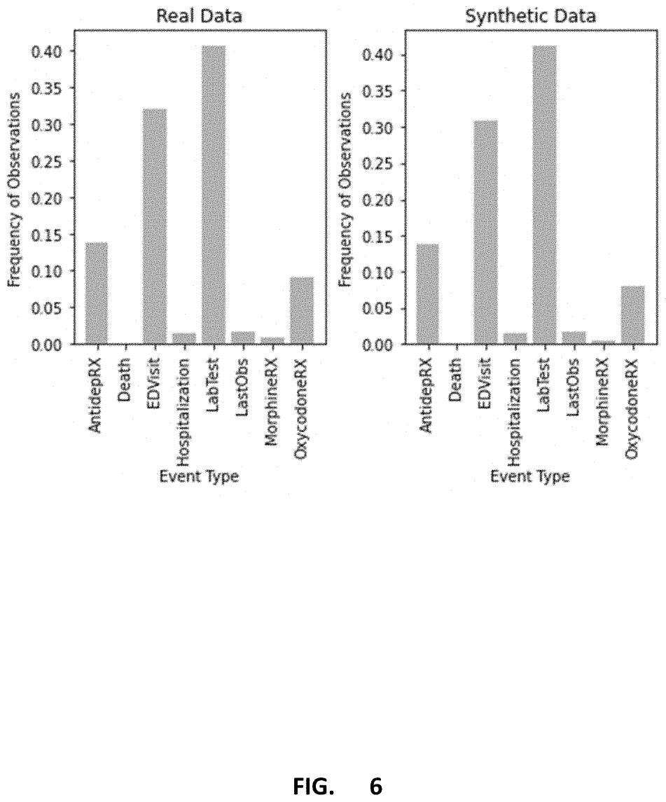

The sequence lengths in the synthetic datasets matched the real dataset quite closely (percent difference in mean sequence length 0.4%) as illustrated in FIG. 5, which depicts a sequence length comparison between the real and synthetic datasets. The distribution of events observed across all synthetic patients matched the distribution of events in the real dataset quite closely (Hellinger distance 0.027) as illustrated in FIG. 6 which depicts an event distribution comparison between the real and synthetic datasets. Overall, the synthetic data has a similar distribution of sequence lengths than in the real data. The real mean & SD was 58.14, 68.57 respectively compared to the synthetic mean & SD of 58.39, 75.16 respectively.

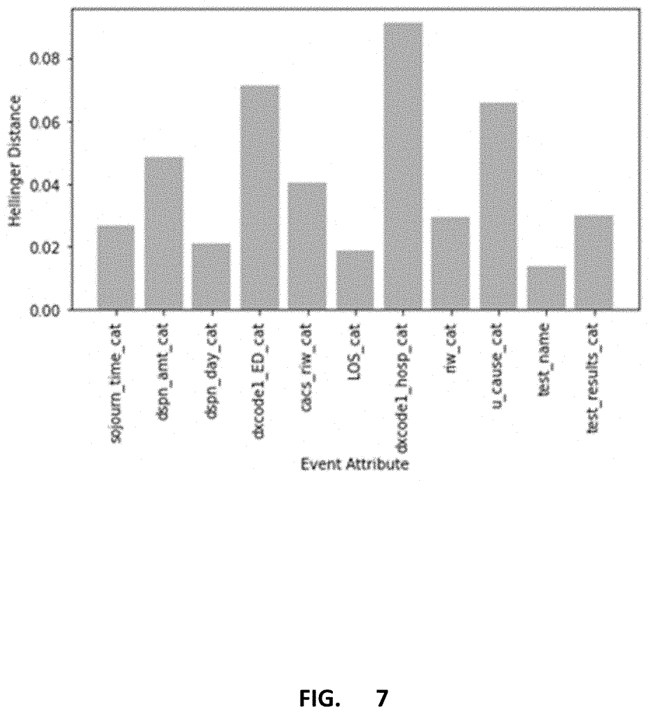

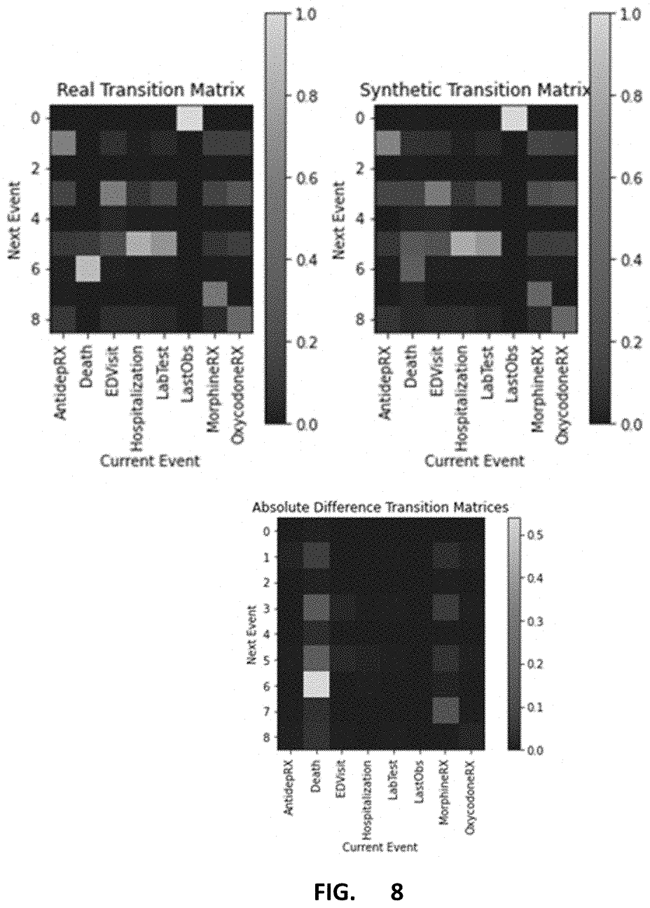

Comparing the distribution of event attributes, the synthetic data again matches the distributions seen in the real data closely as shown in the Hellinger distance histogram in FIG. 7, which depicts the Hellinger distance for each event attribute with a mean Hellinger distance of 0.0417. The differences in the real and synthetic transition matrices was smaller for first order Markov transition matrices as shown in FIG. 8, which depicts heatmaps of first order Markov transition matrices between the real and synthetic datasets, than for second order transition matrices, (mean Hellinger distance 0.0896 vs 0.2195) indicating that short term dependencies may be modelled better than long term dependencies. Note that the heatmaps in FIG. 8 have different scales.

### Workload Aware Assessment

The workload aware assessment of utility was conducted on 75,660 real patient records and 75,660 synthetic records. Standardized mean differences (SMD) indicated that no clinically important differences were noted with respect to demographics and the comorbidity score between the real and synthetic data, shown in Table 8. For example, between the real and synthetic data the mean age was 43.32 vs 44.79 (SMD 0.078), 51.0% males vs 52.5% (SMD 0.029), and Elixhauser comorbidity score of 0.96 vs 1.05 (SMD 0.055). However, differences were noted that would be considered potentially clinically important for laboratory data with standardized mean differences between the real and synthetic data >0.1, a threshold often recommended for declaring imbalance.

TABLE 8
Comparison of trial characteristics across
the real and synthetic datasets.

| | Real | | Synthetic | | |
|---|---|---|---|---|---|
| | n = 75,660 | | n = 75,660 | | SMD |
| Age | | | | | 0.078 |
| Mean (SD) | 43.32 | (17.87) | 44.79 | (19.83) | |
| Median (IQR) | 42.00 | [27.00] | 43.00 | [30.00] | |
| Sex n (%) | | | | | 0.029 |
| Male | 38,623 | (51.0) | 39,711 | (52.5) | |
| Female | 37,037 | (49.0) | 35,949 | (47.5) | |
| Elixhauser | | | | | 0.055 |
| Mean (SD) | 0.96 | (1.58) | 1.05 | (1.63) | |
| Median (IQR) | 0.00 | [1.00] | 0.00 | [2.00] | |
| ALT | | | | | 0.099 |
| Mean (SD) | 31.67 | (63.90) | 40.72 | (111.92) | |
| Median (IQR) | 24.00 | [18.00] | 26.00 | [19.00] | |
| eGFR | | | | | 0.112 |
| Mean (SD) | 85.82 | (23.56) | 83.11 | (25.05) | |
| Median (IQR) | 87.00 | [41.00] | 84.00 | [38.00] | |
| HCT | | | | | 0.291 |
| Mean (SD) | 0.42 | (0.05) | 0.41 | (0.06) | |
| Median (IQR) | 0.42 | [0.05] | 0.41 | [0.06] | |
| CACS-RIW | | | | | 0.002 |
| Mean (SD) | 0.05 | (0.07) | 0.05 | (0.07) | |
| Median (IQR) | 0.03 | [0.03] | 0.03 | [0.03] | |
| RIW | | | | | 0.002 |
| Mean (SD) | 1.40 | (2.73) | 1.40 | (2.40) | |
| Median (IQR) | 0.77 | [0.82] | 0.81 | [0.84] | |
| Opioid Utilization (%) | | | | | |
| Morphine | 1,758 | (2.3) | 2,649 | (3.5) | 0.070 |
| Oxycodone | 73,902 | (97.7) | 73,011 | (96.5) | |
| Antidepressant Use | 28224 | (37.3) | 29651 | (39.2) | 0.039 |

TABLE 9
Outcomes of interest for both real and synthetic datasets.

| | Real | | Synthetic | | |
|---|---|---|---|---|---|
| | N = 75,660 | | N = 75,660 | | SMD |
| Total follow-up | 1,474.48 | (772.23) | 1,077.88 | (722.44) | 0.530 |
| time Mean (SD) | | | | | |
| Mortality | 3,299 | (4.4) | 1,440 | (1.9) | 0.141 |
| n (%) | | | | | |
| Hospitalization | 22,495 | (29.7) | 21,582 | (28.5) | 0.027 |
| n (%) | | | | | |
| Emergency room | 64,376 | (85.1) | 65,193 | (86.2) | 0.031 |
| visit n (%) | | | | | |
| Composite | 64,848 | (85.7) | 65,497 | (86.6) | 0.025 |
| endpoint n (%) | | | | | |
| Diagnosis of | 505 | (2.2) | 472 | (2.2) | 0.004 |
| pneumonia | | | | | |
| (ICD10: J189) | | | | | |
| n (%) | | | | | |

The cumulative follow-up time, post-receipt of the index opioid prescription and the outcomes of interest for the real and synthetic data are summarized in Table 9. Based on SMD cumulative follow-up time (mean of 1,474.48 vs 1,077.88; SMD: 0.530) and mortality (3,299 vs 1,440; SMD: 0.141) yielded a significant difference between the real and synthetic datasets.

TABLE 10
Adjusted hazard ratios and confidence interval overlap for
outcomes of interest in real and synthetic datasets.

| | | | CI-Overlap- |
|---|---|---|---|
| Outcome | Real Data | Synthetic Data | percent |
| Mortality | 0.29 (0.25, 0.33) | 0.35 (0.29, 0.41) | 38% |
| Hospitalization | 0.62 (0.57, 0.67) | 0.64 (0.6, 0.68) | 77% |
| Emergency room | 0.76 (0.71, 0.81) | 0.74 (0.71, 0.78) | 76% |
| visit | | | |
| Composite endpoint | 0.71 (0.66, 0.75) | 0.73 (0.69, 0.77) | 72% |
| Pneumonia | 0.79 (0.5, 1.26) | 0.7 (0.48, 1.03) | 81% |

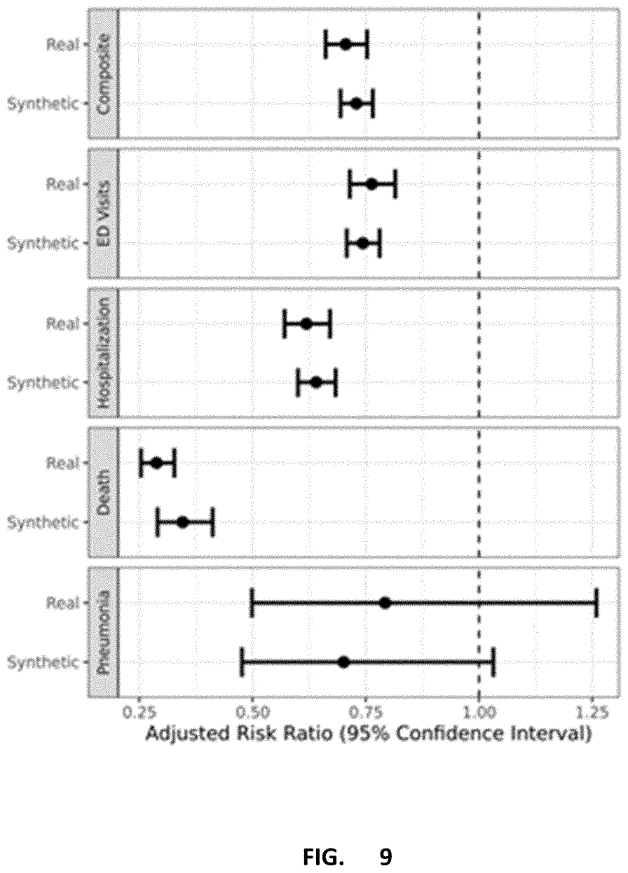

After adjustment for age, sex, use of antidepressants, and laboratory data, the Cox proportional hazards were similar between the real and synthetic datasets. In the real data, oxycodone was associated with a 29% reduction in time to composite endpoint compared to morphine: adjusted HR (aHR) 0.71 95% CI 0.66-0.75). A similar reduction was observed in the synthetic dataset with a 27% reduction in time to event: aHR 0.73 95% CI 0.69-0.77 (FIG. 9 and Table 10). With respect to secondary outcomes, similar trends were observed with minimal differences noted in time to event between the synthetic and real data with the exception of all-cause mortality shown in FIG. 9. With respect to all-cause mortality, although both the real and synthetic data would provide similar conclusions that oxycodone is beneficial on mortality, the estimated effect was higher in the real data, with only a 38% confidence interval overlap (aHR 0.29 (95% CI 0.25, 0.33) vs aHR 0.35 (95% CI 0.29, 0.41)).

The confidence intervals and point estimates in the adjusted Cox regression analysis are also similar and would lead researchers to reach the same conclusion for many applications whether they analyzed real or synthetic datasets. For the adjusted models the mean confidence interval overlap is 68%. This indicates that the conclusions drawn from the synthetic datasets substantially overlap those drawn from the real data.

As described further below, a recurrent neural network model was used for the generation of longitudinal health data from the province of Alberta and evaluated the synthetic longitudinal data utility. Utility is a measure of how similar the results and conclusions are from models built using real and synthetic data.

The model was empirically tested on Alberta's administrative health records. Individuals were selected for this cohort if they received a prescription for an opioid during the 7-year study window. Data available for this cohort of patients includes demographic information, laboratory tests, prescription history, physician visits, emergency department visits, hospitalizations, and death. The analysis used to compare the real data with the synthetic data used traditional time-event analyses that are the cornerstone of most health services research.

Realistic synthetic data for complex longitudinal administrative health records, or other types of data can be generated as described above. Modelling events over time using a form of conditional LSTM allows patterns in the data over time to be learnt, as well as how these trends relate to fixed baseline characteristics. The masking implemented during model training has allowed the data synthesis to work with sparse attribute data from a variety of sources in a single model. Overall, this method of generating synthetic longitudinal health data has performed quite well.

The model learns and recreates patterns in the heterogeneous attributes, accounting for the pattern of relevant attributes based on event type. The generated sequences have event lengths that are consistent with the real data (percent difference in mean sequence length −0.4%). Baseline characteristics were synthesized to be consistent with the distributions in the real data and to exert reasonable influence on the progression of events. This model has been applied to real administrative health data and has performed well on key metrics including confidence interval overlap (mean CI overlap 46%). The process described above has shown the ability of synthetic data to reproduce results of traditional epidemiology analyses. The contrast of the complete dataset to the reduced events dataset synthesis has shown that the best analytic results are produced when the dataset synthesized more closely matches the dataset used in analysis. Removing events not relevant for the planned analysis led to less noise in the dataset, allowing synthesis to reproduce the analytic conclusions better.

This method allows the synthesis of associated cross sectional and longitudinal health data, where the measures included correspond to a variety of medical events (e.g., prescriptions, doctor visits, etc.) and data types (e.g., continuous, categorical). The longitudinal data generated varies in the number of observations per individual, reflecting the structure of real electronic health data. The model selected is easy to train and automatically adapts as the number of events, event attributes, or complexity of attributes changes. The utility of the generated synthetic data was rigorously evaluated using generic and workload aware assessments that have shown the similarity of the generated data to the real data.

The generation of synthetic longitudinal data as described above has generated realistic synthetic data for complex longitudinal administrative health records, although it may be applied to other domains as well. Modelling events over time using a form of conditional LSTM has allowed patterns in the data over time to be learned, as well as how these trends relate to fixed baseline characteristics. The masking implemented during model training has allowed the model to work with sparse attribute data from a variety of sources in a single model. Overall, this method of generating synthetic longitudinal health data has performed quite well from a data utility perspective.

The synthetic longitudinal data generation model as described above may learn and recreate patterns in the heterogeneous attributes, accounting for the pattern of relevant attributes based on event type. The generated sequences have event lengths that are consistent with the real data (percent difference in mean sequence length 0.4%). Baseline characteristics were synthesized to be consistent with the distributions in the real data and to exert reasonable influence on the progression of events. Models as described above have been applied to real administrative health data and have performed well on key metrics including confidence interval overlap (mean CI over 68%). As described herein, it is possible to generate synthetic data that reproduces results of traditional epidemiology analyses.

The data synthesis methodology described herein has worked well with real-world complex longitudinal data that has received minimal curation. This method allows the synthesis of associated cross sectional and longitudinal health data, where the measures included correspond to a variety of medical events (e.g., prescriptions, doctor visits, etc.) and data types (e.g., continuous, categorical). The longitudinal data generated varies in the number of observations per individual, reflecting the structure of real electronic health data. The model selected is easy to train and automatically adapts as the number of events, event attributes, or complexity of attributes changes. The utility of the generated synthetic data, as assessed using generic and workload aware assessments, has similar utility to the real data.

The models for generating the synthetic longitudinal data may use a tabular generative model as an input to the longitudinal generative model. The tabular generative model may use, for example, a sequential tree-based generation method to generate baseline values that reflect the real data. Further, the longitudinal generative module may use masking on the loss function to focus only on the relevant attributes at a particular point in time. During training of the model, the loss for event attributes and event labels may be dynamically weighted. Further, the model may use multiple embedding layers, which allows the model to handle heterogeneous data types.

The above has described systems and methods that may be useful in generating synthetic longitudinal data. Particular examples have been described with reference to clinical trial data. It will be appreciated that, while synthetic data generation may be important in the health and research fields, the above also applies to generating synthetic data in other domains.

Although certain components and steps have been described, it is contemplated that individually described components, as well as steps, may be combined together into fewer components or steps or the steps may be performed sequentially, non-sequentially or concurrently. Further, although described above as occurring in a particular order, one of ordinary skill in the art having regard to the current teachings will appreciate that the particular order of certain steps relative to other steps may be changed. Similarly, individual components or steps may be provided by a plurality of components or steps. One of ordinary skill in the art having regard to the current teachings will appreciate that the components and processes described herein may be provided by various combinations of software, firmware and/or hardware, other than the specific implementations described herein as illustrative examples.

The techniques of various embodiments may be implemented using software, hardware and/or a combination of software and hardware. Various embodiments are directed to apparatus, e.g. a node which may be used in a communications system or data storage system. Various embodiments are also directed to non-transitory machine, e.g., computer, readable medium, e.g., ROM, RAM, CDs, hard discs, etc., which include machine readable instructions for controlling a machine, e.g., processor to implement one, more or all of the steps of the described method or methods.

Some embodiments are directed to a computer program product comprising a computer-readable medium comprising code for causing a computer, or multiple computers, to implement various functions, steps, acts and/or operations, e.g. one or more or all of the steps described above. Depending on the embodiment, the computer program product can, and sometimes does, include different code for each step to be performed. Thus, the computer program product may, and sometimes does, include code for each individual step of a method, e.g., a method of operating a communications device, e.g., a wireless terminal or node. The code may be in the form of machine, e.g., computer, executable instructions stored on a computer-readable medium such as a RAM (Random Access Memory), ROM (Read Only Memory) or other type of storage device. In addition to being directed to a computer program product, some embodiments are directed to a processor configured to implement one or more of the various functions, steps, acts and/or operations of one or more methods described above. Accordingly, some embodiments are directed to a processor, e.g., CPU, configured to implement some or all of the steps of the method(s) described herein. The processor may be for use in, e.g., a communications device or other device described in the present application.

Numerous additional variations on the methods and apparatus of the various embodiments described above will be apparent to those skilled in the art in view of the above description. Such variations are to be considered within the scope.

What is claimed is:

1. A method for synthesizing longitudinal data comprising:
generating baseline characteristics and first event values for a plurality of synthetic individuals using a trained model;
for each synthetic individual in the generated baseline characteristics, generating a plurality of sequential event values by iteratively:
using a trained model, predicting a next event comprising an event label and associated event attributes based on previous events for the respective synthetic individual; and
masking from the predicted next event any predicted associated event attributes based on an attribute mask associated with the event label of the predicted next event; and
outputting a synthetic data set comprising the synthesized baseline characteristics, first event values and synthesized sequential events of the plurality of synthetic individuals.

2. The method of claim 1, wherein the trained model for synthesizing the baseline characteristics and first event values uses a sequential tree-based method.

3. The method of claim 1, wherein the trained model used for predicting a next event comprises a long short term memory (LTSM) model.

4. The method of claim 1, wherein each event label is predicted from a predefined set of event labels.

5. The method of claim 4, wherein the trained model used for predicting a next event further comprises a first embedding layer for mapping event labels to a series of continuous features that are provided as input to the LTSM model.

6. The method of claim 5, wherein the trained model used for predicting a next event further comprises a second embedding layer for mapping event attributes to a series of continuous features that are provided as input to the LTSM model.

7. The method of claim 1, wherein each of the plurality of sequential events are associated with an event time of occurrence.

8. The method of claim 1, further comprising training the model used to synthesize baseline characteristics and first event values from real longitudinal data.

9. The method of claim 1, further comprising training the model used to synthesize the plurality of sequential event values using real longitudinal data.

10. The method of claim 1, wherein the longitudinal data comprises health data.

11. A non-transitory computer readable memory, which when executed configure a computing system to implement a method for synthesizing longitudinal data, the method comprising:
generating baseline characteristics and first event values for a plurality of synthetic individuals using a trained model;
for each synthetic individual in the generated baseline characteristics, generating a plurality of sequential event values by iteratively:
using a trained model, predicting a next event comprising an event label and associated event attributes based on previous events for the respective synthetic individual; and
masking from the predicted next event any predicted associated event attributes based on an attribute mask associated with the event label of the predicted next event; and
outputting a synthetic data set comprising the synthesized baseline characteristics, first event values and synthesized sequential events of the plurality of synthetic individuals.

12. The non-transitory computer readable memory of claim 11, wherein the trained model for synthesizing the baseline characteristics and first event values uses a sequential tree-based method.

13. The non-transitory computer readable memory of claim 11, wherein the trained model used for predicting a next event comprises a long short term memory (LTSM) model.

14. The non-transitory computer readable memory of claim 11, wherein each event label is predicted from a predefined set of event labels.

15. The non-transitory computer readable memory of claim 14, wherein the trained model used for predicting a next event further comprises a first embedding layer for mapping event labels to a series of continuous features that are provided as input to the LTSM model.

16. The non-transitory computer readable memory of claim 15, wherein the trained model used for predicting a next event further comprises a second embedding layer for mapping event attributes to a series of continuous features that are provided as input to the LTSM model.

17. The non-transitory computer readable memory of claim 11, wherein each of the plurality of sequential events are associated with an event time of occurrence.

18. The non-transitory computer readable memory of claim 11, wherein the method provided by executing the instructions stored on the non-transitory computer readable memory further comprises training the model used to synthesize baseline characteristics and first event values from real longitudinal data.

19. The non-transitory computer readable of claim 11, wherein the method provided by executing the instructions stored on the non-transitory computer readable memory further comprises training the model used to synthesize the plurality of sequential event values using real longitudinal data.

20. The non-transitory computer readable of claim 11, wherein the longitudinal data comprises health data.

21. A system for synthesizing longitudinal data comprising:
a processor for executing instructions; and
a memory storing instructions which when executed configure the system to implement a method for synthesizing longitudinal data, the method comprising:
generating baseline characteristics and first event values for a plurality of synthetic individuals using a trained model;
for each synthetic individual in the generated baseline characteristics, generating a plurality of sequential event values by iteratively:
using a trained model, predicting a next event comprising an event label and associated event attributes based on previous events for the respective synthetic individual; and
masking from the predicted next event any predicted associated event attributes based on an attribute mask associated with the event label of the predicted next event; and
outputting a synthetic data set comprising the synthesized baseline characteristics, first event values and synthesized sequential events of the plurality of synthetic individuals.
